(19) United States
(12) Plant Patent Application Publication
(10) Pub. No.: US 2023/0157189 P1
(43) Pub. Date: May 18, 2023

(54) Apple Tree Rootstock Named 'D2212'

(50) ;

(71) Applicant: Bundesrepublik Deutschland vertreten durch das Julius Kühn-Institut, Quedlinburg (DE)

(72) Inventor: Erich Seemüller, Dossenheim (DE)

(21) Appl. No.: 17/963,246

(22) Filed: Oct. 11, 2022

Related U.S. Application Data
(60) Provisional application No. 63/361,051, filed on Nov. 18, 2021.

(51) Int. Cl. A01H 6/74

(52) U.S. Cl. PLT/174

(57) ABSTRACT

A new and distinct variety of apple tree rootstock that exhibits compatibility with all apple varieties and resistance to Candidatus Phytopiasma mali and Athelia rolfsii.

### CROSS REFERENCE TO RELATED APPLICATION(S)

This application claims the benefit of priority from U.S. Provisional Patent Application No. 63/361,051, filed Nov. 18, 2021, which is incorporated herein by reference in its entirety.

Botanical classification: Malus mill.

Varietal denomination: ‘D2212’

### BACKGROUND OF THE NEW PLANT

The present invention comprises a new and distinct variety of apple tree that is solely used as a rootstock and known by the varietal name ‘D2212’. The new variety was developed by the breeder as part of a planned breeding program in Ahrensburg, Germany. The purposes of the breeding program were to obtain rootstocks with resistance to Candidatus Phytoplasma maii and with reduced plant vigor. ‘D2212’ is the result of open pollination of an unpatented variety of M. domestica x M. sieboldii named ‘Laxton's Superb’ (female parent). As a result, the exact male parent is unknown. The new variety was selected and first asexually reproduced by in-vitro propagation in Stuttgart, Germany.

When compared to unpatented Malus mill. varieties referred to as PJ-P 21 (APU 85) and M 25 (APU 29), ‘D2212’ has crenate leaf blade margin incisions, while the comparison varieties have serrate leaf blade margin incisions. Further, ‘D2212’ has weak pubescence on the lower side of its leaf blades, while M 25 (APU 29) has strong pubescence on the lower side of its leaf blades. Also, resistance to Candidatus Phytoplasma mali and Athelia rolfsii are distinguishing characteristics of the new variety from other apple tree varieties known to the breeder.

The new variety has not been evaluated under all possible environmental conditions. The phenotype may vary with variations in environment without a change in the genotype of the plant. The new variety has been trial and field tested and has been found to retain its distinctive characteristics and remain true to type through successive asexual propagations.

### DESCRIPTION OF THE DRAWINGS

The accompanying photographic drawings illustrate the new variety at with the color being as nearly true as possible with color illustrations of this type.

FIG. 1 illustrates multiple plants of the new variety at approximately 1 year of age;

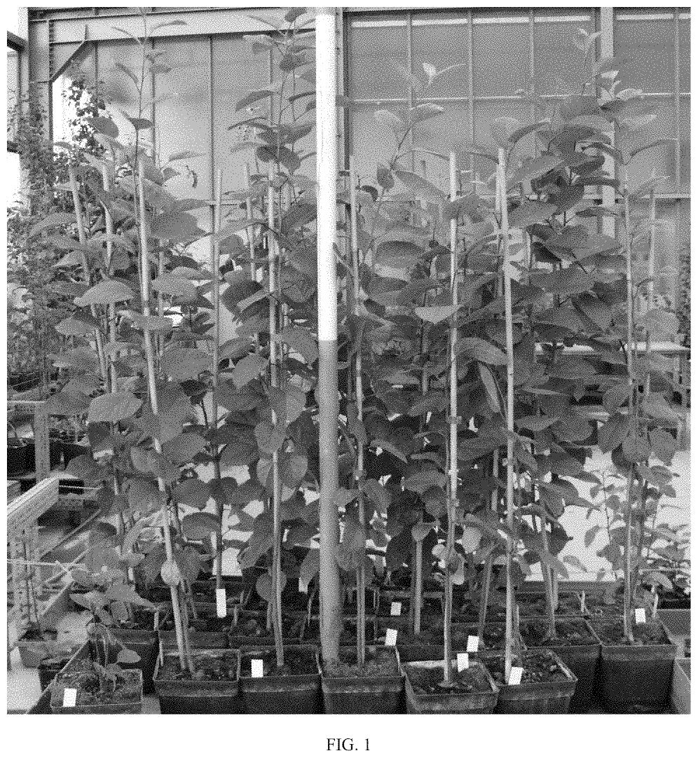

FIG. 2 illustrates multiple young plants of the new variety; and

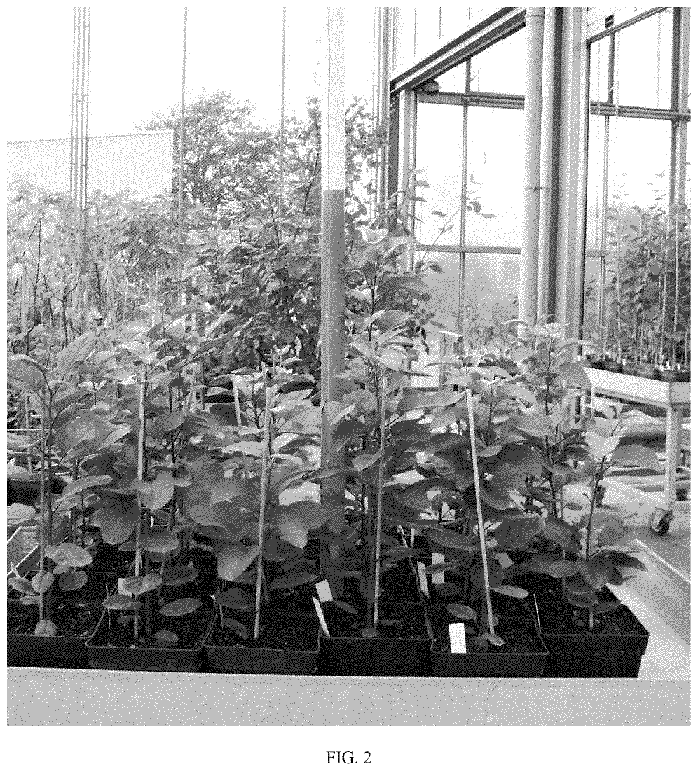

FIG. 3 illustrates the new variety at 7 years of age with fruits thereon.

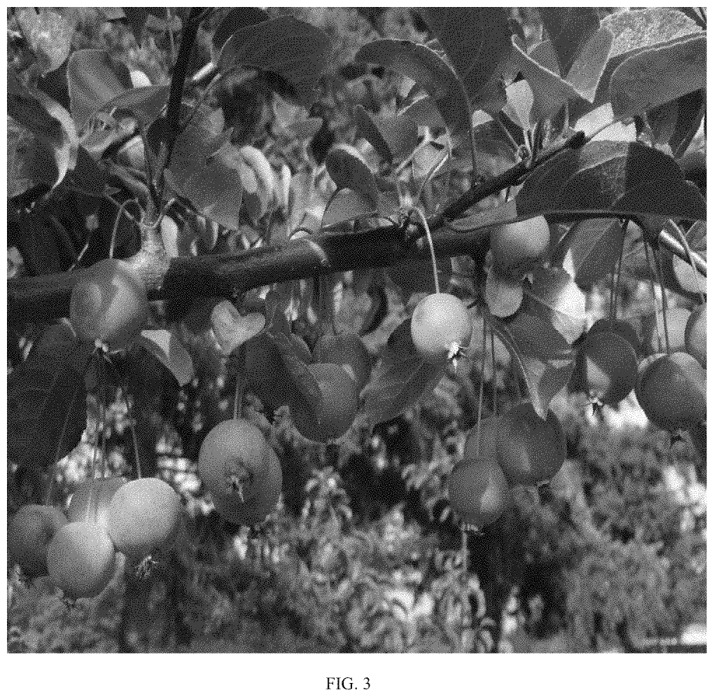

### DETAILED BOTANICAL DESCRIPTION

The following detailed description sets forth the characteristics of the new variety at approximately 1 year of age and on a mother plant at 7 years of age. The data which defines these characteristics were collected as a result of asexual reproductions carried out in Rheinstetten, Germany on potted plants under standard growing conditions and taken under natural light. Where applicable, color descriptions are provided using X11 Color Names and their corresponding HEX Color Codes, accessed in August of 2022 via https://www.farbtabelle.de/de/farbtabelle.htm.

### ROOTSTOCK

Height: 150.0 cm.
Thickness: 12.0 mm.
Vigor: Medium.
Tree habit: Upright and straight growth.
Form: Spindle.
Use: Solely as a rootstock.
Multiplication ability:
   Layering.—Not determined to date.
   Hardwood cuttings.—Very difficult.
   Softwood cuttings.—Possible, but with low yields.
Pathogen resistance: Resistant to Candidatus Phytoplasma mali and Athelia rolfsii.
Pest resistance/susceptibility: Not observed to date.
Rootstock performance:
   Root sprouts (suckering).—None.
   Anchorage.—Stable in the ground.
   Compatibility.—Compatible with all varieties.
   Vigor.—Medium to strong.
Shoot:
   Diameter.—Thick at midpoint.
   Internode length.—Medium to long.
   Texture.—Pubescence present on upper half of shoot.
   Glossiness of bark.—Medium to strong.
   Color.—Reddish brown on sunny side.
   Growing tip color.—Reddish.
Buds:
   Position.—Adpressed relative to axis.
   Size.—Medium.
   Shape of tip.—Pointed.
   Support size.—Small to medium.
Lenticels:
   Quantity.—Average.
   Length.—Medium to long.
   Shape.—Elliptic.
Petiole:
   Shape.—Straight.
   Length.—2.0 cm; 3.5 length of blade to length of petiole ratio.
   Diameter.—0.15 cm.
Leaves:
   Arrangement.—Alternate, spiral.
   Length.—From 6.5 to 7.0 cm.
   Width.—From 5.0 to 6.0 cm.
   Form.—Generally, elliptic-oval. Lower base leaves are often triangular with basal lobes.
   Profile.—Concave in cross section.
   Apex.—Pointed.
   Base.—Orbicular-obtuse.
   Margin.—Double saw incisions present.
   Texture.—Upper surface is smooth; lower surface has weak pubescence present.
   Color.—Young leaves have bronze anthocyanin coloration present.
   Venation.—Type: Pinnate, alternate. Color: Slightly reddish with very weak to weak anthocyanin coloration present.
Flower buds:
   Pedicel.—Length: 4.5 cm. Diameter: 1.0 mm. Color: Tan4, HEX #8b5a2b.
   Bud.—Length: 1.0 cm. Width: 4.0 mm. Color: HotPink, HEX #ff69b4.
Flowers:
   Bloom timing.—From April 23 to May 8 in Rheinstetten, Germany.
   Blooming period.—Two weeks.
   Number of flowers per cluster.—4 to 6.
   Fragrance.—Weakly sweet.
   Petals.—Number: 5. Length: 2.5 cm. Width: 1.7 cm. Shape: Orbicular. Margin: Entire. Texture: Smooth.
   Color.—When opening: Upper surface: Pink1, HEX #eea9b8. Lower surface: MistyRose, HEX #ffe4e1. Fully opened: Upper surface: White, HEX #ffffff. Lower surface: LightPink1, #ffaeb9.
   Sepals.—Shape: Rhomboid. Margin: Entire. Texture: Smooth. Length: 4.0 to 5.0 mm. Width: 1.0 mm. Color: Upper surface: OliveDrab4, HEX #698b22. Lower surface: OliveDrab3, HEX #9acd32.
Reproductive organs:
   Stamens.—Number (per flower): 12. Filament length: 3.0 mm.
   Anthers.—Shape: Round. Length: 1.0 mm. Color: Gold3, HEX #cdad00.
   Pollen.—Color: Gold3, HEX #cdad00.
   Pistils.—Length: 2.4 cm.
   Style.—Length: 7.0 mm. Color: Cornsilk3, HEX #cdc8b1.
   Stigma.—Shape: Round. Color: NavajoWhite3, HEX #cdb38b.
Fruit: Not observed to date.

I claim:

1. A new and distinct variety of apple tree rootstock plant named ‘D2212’, as is herein illustrated and described.
